(19) United States
(12) Patent Application Publication
(10) Pub. No.: US 2015/0111764 A1
(43) Pub. Date: Apr. 23, 2015

(54) POLYMERIZED MICROARRAYS

(71) Applicant: New York University, New York, NY (US)

(72) Inventors: Adam B. Braunschweig, Miami, FL (US); Shudan Bian, Coral Gables, FL (US); Sylwia Zieba, New York, NY (US)

(73) Assignee: New York University, New York, NY (US)

(21) Appl. No.: 14/059,788

(22) Filed: Oct. 22, 2013

(51) Int. Cl. B01J 19/00; G01N 33/68

(52) U.S. Cl. CPC B01J 19/0046; G01N 33/6842; B01J 2219/00731; B01J 2219/00711; B01J 2219/00675; B01J 2219/00637; B01J 2219/00621
USPC 506/9; 506/19; 506/32

(57) ABSTRACT

Micropatterns of glycan-bearing brush polymers generated by the initiation of oligomerization of acrylate and methacrylate monomers from thiol-terminated surfaces. Chain lengths are controlled in situ by varying exposure time, and these multivalent glycan scaffolds detect glycan binding proteins at sub-micromolar concentrations.

### STATEMENT OF GOVERNMENT INTEREST

The United States Government has rights in the invention described herein pursuant to an Air Force Office of Scientific Research Young Investigator Award FA9550-11-1-0032 and the National Science Foundation grant DBI-115269 and DBI-1340038.

### FIELD OF THE INVENTION

The present invention generally relates glycan microarrays.

### BACKGROUND OF THE INVENTION

Glycan microarrays, composed of carbohydrates patterned onto substrates, have become an invaluable tool for investigating the role of glycans and glycan binding proteins (GBPs) on cell adherence, motility, and signaling processing, which has important implications for therapeutics and diagnostics. Because glycan recognition occurs on cell surfaces, the 3D structure of the carbohydrates plays a critical role on specificity and binding affinity as a result of the multivalent recognition that dominates the interactions between glycans and GBPs. However, comparing the binding to glycan arrays prepared by different methods is difficult because of this sensitivity of GBP binding affinity to carbohydrate orientation and density, and vastly different results have been obtained depending on the deposition method used in preparing the arrays. To achieve uniform and reproducible glycan deposition, significant efforts have been devoted to exploring new, highly specific covalent chemistries for carbohydrate immobilization, including the Cu^{I}-catalyzed azide alkyne click reaction, hydrazide formation, and photochemical approaches, that are compatible with the functional groups common to carbohydrates. A drawback of these methods is that for high detection sensitivity, multivalent glycans must be first prepared and appropriately functionalized to undergo the surface reactions, but these multistep syntheses of dendronized carbohydrates are complex and challenging. Thus the ideal chemistry for preparing glycan arrays should use simple starting materials, bioorthogonal reactions, be compatible with molecular printing approaches, and produce complex 3D scaffolds to access multivalent binding.

### SUMMARY OF THE INVENTION

One embodiment of the invention relates to a microarray comprising a thiol-terminated substrate and a plurality of polymer brushes bound via thiol-acrylate polymerization to the thiol-terminated substrate.

Another embodiment relates to a method of making a microarray. A substrate is provided having thiol associated therewith. A (meth)acrylate-containing monomer is deposited on the substrate. An initiator is deposited on the substrate. The substrate is irradiated with the deposited (meth)acrylate-containing monomers and photoinitiator. Thiol-(meth)acrylate polymerization is induced. An oligomer comprising the (meth)acrylate-containing monomers is bound to the substrate.

Another embodiment relates to a method of investigating glycans and glycan binding proteins. A substrate having thiol associated therewith is provided. A glycan-modified (meth)acrylate monomer is deposted on the substrate. A thiol-acrylate polymerization is induced. A plurality of polymer brushes is bound to the substrate. The plurality of polymer brushes provide sufficient glycan density to access multivalent glycan binding protein modes.

Additional features, advantages, and embodiments of the present disclosure may be set forth from consideration of the following detailed description, drawings, and claims. Moreover, it is to be understood that both the foregoing summary of the present disclosure and the following detailed description are exemplary and intended to provide further explanation without further limiting the scope of the present disclosure claimed.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIGS. 1a-b illustrate: FIG. 1(a) a process for inducing the thiol-ene and thiol-(methy)acrylate reaction on the surface by beam pen lithography (BPL). i) The tip-array coated with an ink mixture (blue) containing the probe molecules and PEG matrix was used to deposits the ink mixture onto surface. ii) Light goes through the tip of the beam pens and locally expose the patterned surface. iii) Following rinsing of the surface to remove excess ink, only the covalently immobilized molecules remain on the surface and in FIG. 1(b) molecular probes compound 1 (Rhodamine-acrylate), compound 2 (Rhodamine-thiol), compound 3 (α-glucomethacrylate), compound 4 (glucose alkene), compound 5 (mannose alkene), compound 6 (ferrocene acrylate), and compound 7 (ferrocene alkene) were used to study the BPL and PPL-thiol-ene and thiol-acrylate surface reactions.

FIGS. 2a-f illustrate: FIG. 2(a) a Fluorescence microscopy image (Nikon Eclipse Ti, λₑₓ=532-587 nm, λₑₘ=608-687 nm) of an array of compound 2 patterned by PPL on a alkene-terminated glass slide with dwell times of 50, 500, 5,000, 50,000 and 100,000 ms. Inset shows the pattern prepared by a single tip; 2(b) the fluorescence intensity of features printed with varying dwell times; 2(c) is a graph of dwell time vs feature diameter for the array of FIG. 2a; 2(d) Fluorescence microscopy image (λₑₓ=532-587 nm, λₑₘ=608-687 nm) of an array of compound 1 patterned by PPL on an thiol-terminated glass slide with dwell times of 50, 500, 5000, 50000 and 100000 ms, where the inset shows the pattern prepared by a single tip; and 2(e) the Fluorescence intensity of features printed with varying dwell times; 2(f) is a graph of dwell time vs feature diameter for the array of FIG. 2d.

FIGS. 3a-b illustrate: 3(a) a CV (0.05, 0.10, 0.15, 0.20, 0.25, 0.30 mV/s from black to yellow) of compound 7 on Au using a Pt counter electrode and Ag/AgCl reference electrode in 0.1 M HClO₄ electrolyte. Inset: the relationship between Ln(scan rate) and Ln(current)(Slope is 1); 3(B) a CV (0.10, 0.15, 0.20, 0.25, 0.30 mV/s from blue to purple) of compound 6 on Au using a Pt counter electrode and Ag/AgCl reference electrode in 0.1 M HClO₄ electrolyte, wherein the inset shows the relationship between Ln(scan rate) and Ln(current)(Slope is 0.7).

FIGS. 4a-c (3) illustrate: 4(a) Fluorescence image (Nikon Eclipse Ti, λₑₓ=532-587 nm, λₑₘ=608-687 nm) of an array of compound 1 printed by BPL with illumination times of 2, 5, 10, and 20 min, with the inset showing an array prepared by a single tip; 4(b) an intensity profile of the features printed with different illumination times indicated by the white line in 4(a) with the inset showing the cartoon image of the polymers formed on the surface; FIG. 4(c) The relationship between exposure time and fluorescence intensity or height.

FIG. 5(a) (4) Fluorescence image (Nikon Eclipse Ti, λex=532-587 nm, λem=608-687 nm) of an array of 3 printed by BPL with illumination times of 2, 5, 10, and 20 min. Inset shows an array prepared by a single tip; FIG. 5(b) Binding of ConA to glycan arrays prepared using different surface chemistries and technologies, including α-mannose immobilized by the Cuaac reaction by PPL (green), α-glucose by the thiol-acrylate reaction of 3 by BPL(red), thiol-ene of α-mannose alkene by PPL (blue), thiol-ene reaction with 5 by PPL (purple), and binding of the thiol-acrylate surface of 3 with PNA (black) by PPL.

FIG. 6(a) (5) AFM height image of a 4*4 patterns of 3 bound to Cy3-ConA (b) The height profile of the features printed with different illumination times indicated by the white line in (a). (c) Cartoon image shows the height increases after Cy3-ConA was bound to the sugar. (d) Binding of Cy3-ConA to glycan arrays prepared using surface initiated thiol-acrylate polymerization by BPL with different UV exposure time, 20 min (black), 10 min (red), 5 min (blue) and 2 min (green).

FIG. 7 illustrates a scheme for preparation of compound 1 and compound 2.

FIG. 8 illustrates a scheme for preparation of compound 3, compound 4, compound 6 and compound 7.

FIG. 9 illustrates a ¹H NMR spectrum of compound 4.

FIG. 10 illustrates a ¹³C NMR spectrum of compound 4.

FIG. 11 illustrates a high resolution mass spectrum of compound 4.

FIG. 12 illustrates a scheme for preparation of (a) thiol-terminated glass surfaces, (b) alkene-terminated glass surfaces, and (c) thiol-terminated Au surfaces.

FIG. 13 illustrates the influence of exposure time on the normalized fluorescence intensity of a surface patterned with compound 4 and subsequently exposed to Cy3-labelled ConA.

FIGS. 14a-d illustrate: 13(A) a Fluorescence microscopy image (Nikon Eclipse Ti, λex=532-587 nm, λem=608-687 nm) of an array of compound 1 patterned by PPL on a bare glass slide with dwell times of 50, 500, 5000, 50000 and 100000 ms before washing and 13(b) after washing. Fluorescence microscopy image (λₑₓ=532-587 nm, λₑₘ=608-687 nm) of an array of compound 1 patterned by PPL on an thiol-terminated glass slide (no UV exposure) before washing 13(c) and 13(d) after washing.

FIGS. 15a-d illustrate: 15(a) a Fluorescence microscopy image (Nikon Eclipse Ti, λₑₓ=532-587 nm, λₑₘ=608-687 nm) of an array of compound 2 patterned by PPL on a bare glass slide with dwell times of 50, 500, 5000, 50000 and 100000 ms before washing and 15(b) after washing; 15(c) a fluorescence microscopy image (λₑₓ=532-587 nm, λₑₘ=608-687 nm) of an array of compound 2 patterned by PPL on an alkene-terminated glass slide (no UV exposure) with dwell times of 50, 500, 5000, 50000 and 100000 ms before washing and 15(d) after washing.

FIGS. 16a-b illustrate Cyclic voltammograms (0.1M HClO₄ electrolyte solution, Ag/AgCl reference electrode, Pt counter electrode, at 0.15 V/s scan rate) of compound 6 patterned onto an Au surface (17a) and compound 7 patterned onto an Au surface (17b). The red line demarcates the base line of the CV of the fc bearing Au surface used to calculate Γ_{fc}.

FIGS. 17a-d illustrate: 18(a) an optical microscope image showing PPL-patterned dot arrays of nanoreactors containing compound 6, DMPA and PEG on the pure Au surface with 10 s dwell time; 18(b) a cyclic Voltammetry (CV) characterization of compound 6-bearing pure Au in 18(a) after UV exposure using a Pt counter electrode and Ag/AgCl/1M KCl reference electrode in 0.1M HClO₄(aq); different curves indicate different scan rates (0.30, 0.40, 0.50 V/s from top to bottom); 18(c) an optical microscope image showing PPL-patterned dot arrays of nanoreactors containing compound 7, DMPA and PEG on the thiol-terminated Au surface with 10 s dwell time; 18(d) acyclic Voltammetry (CV) characterization of compound 7-bearing pure Au in 18(c) without UV exposure using a Pt counter electrode and Ag/AgCl/1M KCl reference electrode in 0.1M HClO₄(aq). Different colored curves indicate different scan rates (0.10, 0.20, 0.30, 0.40 V/s from black to green).

FIG. 18 illustrates a fluorescence microscopy image (Nikon Eclipse Ti, λex=532-587 nm, λem=608-687 nm) of an array of 1 patterned by BPL on a thiol-terminated glass slide with 1 s dwell time (no UV exposure) after washing.

FIGS. 19a-b illustrates: 20(a) a fluorescence microscopy image (Nikon Eclipse Ti, λₑₓ=532-587 nm, λₑₘ=608-683 nm) of a surface patterned with 3 and exposed to a solution of 2.17×10⁻⁵ M Cy3-modified ConA, where the inset is a magnified image of a single 4×4 array; 20(b) is an intensity profile of the white line in 20(a).

FIGS. 20a-d illustrate: 21(a) a Fluorescence microscopy image (Nikon Eclipse Ti, λₑₓ=532-587 nm, λₑₘ=608-683 nm) of a surface patterned with compound 3 and exposed to a solution of 1.08×10⁻⁵M Cy3-modified ConA, where the inset is a magnified image of a single 4×4 array; 21(b) an intensity profile of the white line in 21(a); 21(c) a Fluorescence microscopy image (Nikon Eclipse Ti, λₑₓ=532-587 nm, λₑₘ=608-683 nm) of a surface patterned with compound 3 and exposed to a solution of 5.4×10⁻⁶ M Cy3-modified ConA where the inset is a magnified image of a single 4×4 array; 21(d) an intensity profile of the white line in 21(c).

FIGS. 21a-d illustrate: 22(a) a Fluorescence microscopy image (Nikon Eclipse Ti, λₑₓ=532-587 nm, λₑₘ=608-683 nm) of a surface patterned with compound 3 and exposed to a solution of 3.0×10⁻⁶ M Cy3-modified ConA where the inset is a magnified image of a single 4×4 array; 22(b) Intensity profile of the white line in 22(a); 22(c) a Fluorescence microscopy image (Nikon Eclipse Ti, λₑₓ=532-587 nm, λₑₘ=608-683 nm) of a surface patterned with compound 5 and exposed to a solution of 1.7×10⁻⁶ M Cy3-modified ConA where the inset is a magnified image of a single 4×4 array; 22(d) is an intensity profile of the white line in 22(c).

FIGS. 22a-b illustrate: 23(a) a Fluorescence microscopy image (Nikon Eclipse Ti, λₑₓ=532-587 nm, λₑₘ=608-683 nm) of a surface patterned with compound 3 and exposed to a solution of 4.3×10⁻⁷ M Cy3-modified Con; 23(b) an intensity profile of the white line in 23(a).

FIGS. 23a-b illustrate: 24(a) a fluorescence microscopy image (Nikon Eclipse Ti, λₑₓ=532-587 nm, λₑₘ=608-683 nm) of a surface patterned with compound 4 and exposed to a solution of 2.17×10⁻⁵ M Cy3-modified ConA, where the inset is a magnified image of a single 4×4 array; 24(b) an intensity profile of the white line in 24(a).

FIGS. 24a-b illustrate: 25(a) Fluorescence microscopy image (Nikon Eclipse Ti, λₑₓ=532-587 nm, λₑₘ=608-683 nm) of a surface patterned with compound 5 and exposed to a solution of 2.17×10⁻⁵ M Cy3-modified ConA. The inset is a magnified image of a single 4×4 array; 25(b) Intensity profile of the white line in 25(A).

FIGS. 25a-d illustrate: 26(a) Fluorescence microscopy image (Nikon Eclipse Ti, λₑₓ=532-587 nm, λₑₘ=608-683 nm) of a surface patterned with compound 5 and exposed to a solution of 1.08×10⁻⁵ M Cy3-modified ConA where the inset is a magnified image of a single 4×4 array; 26(b) an intensity profile of the white line in 26(a); 26(c) a Fluorescence microscopy image (Nikon Eclipse Ti, λₑₓ=532-587 nm, λₑₘ=608-683 nm) of a surface patterned with compound 5 and exposed to a solution of 5.4×10⁻⁶ M Cy3-modified ConA where the inset is a magnified image of a single 4×4 array; 26(d) is an intensity profile of the white line in 26(c).

FIG. 26 illustrate: 27(a) an optical image of the printing of compound 3 in a PEG matrix on a thiol-terminated glass slide by PPL-induced thiol-acrylate reaction. 27(B) a Fluorescence image of compound 3 on a thiol-terminated glass slid after Rhodamine-PNA interaction; 27(c) an optical image of the printing of compound 4 in a PEG matrix on a thiol-terminated glass slide by PPL-induced thiol-alkene reaction; 27(d) a Fluorescence image of compound 4 on a thiol-terminated glass slide after immersion in a Rhodamine-labeled PNA solution

FIGS. 27a-b illustrate: 27(a) an AFM tapping mode image after washing of a 4×4 dot array of compound 1 patterned onto the thiol-terminated glass slide and exposed to UV/light; 27(b) an AFM 3D image of the 4×4 dot array in 27(a).

FIG. 28 illustrates: (a) Fluorescence microscopy image (Nikon Eclipse Ti, λₑₓ=532-587 nm, λₑₘ=608-683 nm) of a surface patterned with 3 and exposed to a solution of 21.7×10⁻⁶ M Cy3-modified ConA. The inset is a magnified image of a single 4×4 array. b) Intensity profile of the white line in (a).

FIG. 29 illustrates: (a) Fluorescence microscopy image (Nikon Eclipse Ti, λₑₓ=532-587 nm, λₑₘ=608-683 nm) of a surface patterned with 3 and exposed to a solution of 5.4×10⁻⁶ M Cy3-modified ConA. The inset is a magnified image of a single 4×4 array. b) Intensity profile of the white line in (a). (c) Fluorescence microscopy image (Nikon Eclipse Ti, λₑₓ=532-587 nm, λₑₘ=608-683 nm) of a surface patterned with 3 and exposed to a solution of 3.0×10⁻⁶ M Cy3-modified ConA. The inset is a magnified image of a single 4×4 array. d) Intensity profile of the white line in (c).

FIG. 30 illustrates: (a) Fluorescence microscopy image (Nikon Eclipse Ti, λₑₓ=532-587 nm, λₑₘ=608-683 nm) of a surface patterned with 3 and exposed to a solution of 1.7×10⁻⁶ M Cy3-modified ConA. The inset is a magnified image of a single 4×4 array. b) Intensity profile of the white line in (a). (c) Fluorescence microscopy image (Nikon Eclipse Ti, λₑₓ=532-587 nm, λₑₘ=608-683 nm) of a surface patterned with 3 and exposed to a solution of 4.3×10⁻⁷ M Cy3-modified ConA. The inset is a magnified image of a single 4×4 array. d) Intensity profile of the white line in (c).

FIG. 31 illustrates: (a) Fluorescence image of 3 immobilized on a thiol-terminated glass slide after immersion in a Rhodamine-labeled PNA solution. (b) Fluorescence image of 4 immobilized on a thiol-terminated glass slide after immersion in a Rhodamine-labeled PNA solution.

FIG. 32 illustrates: (a) Fluorescence image of 5 immobilized on a thiol-terminated glass slide after immersion in a Cy3-ConA solution. (b) Intensity profile of the white line in (a). (c) Fluorescence image of 3 immobilized on a thiol-terminated glass slide after immersion in a Cy3-ConA solution.

### DETAILED DESCRIPTION OF THE PREFERRED EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

One implementation of the invention relates to glycan arrays and methods for preparing the same. In one implementation, glycan arrays are prepared by the photoinitiated polymerization of glycan functionalized methacrylate and acrylate monomers grafted from thiol-terminated glass and Au surfaces. In another implementation, the glycan arrays are initiated thermally. In one implementation, Michael addition is utilized or radical initiator like benzoyl peroxide or AIBN is utilized. Although the thiol-ene radical reaction, which brings together thiols and alkenes under UV irradiation, has been used successfully to prepare microarrays, including glycan patterns, thiol-acrylate reactions have not be utilized for microarrays.

In one implementation, a carbohydrate-modified methacrylate and thiol-terminated glass combine via a thiol-acrylate polymerization. The resultant formation of oligomeric carbohydrates that protrude from the surface of the substrate with the 3D structure and monosaccharide density necessary to access multivalent GBP binding modes. This contrasts with the structure of a thiol-ene reaction based microarray where the structures extend a shorter distance from the substrate and do not provide access to multivalent binding modes. The basic thiol-ene reaction and the basic thiol-acrylate reaction of certain implementations of the present invention are show below.

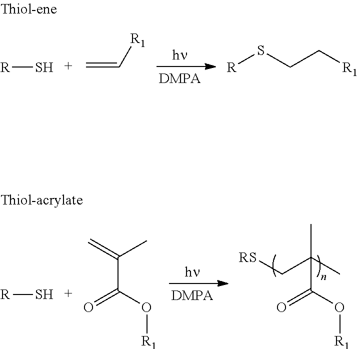

In one implementation the (meth)acrylate-group may be part of a polymer brush. Polymer brushes have been investigated widely for tailoring surface functionality and morphology, but their utility in glycan, glycan binding protein, antibody, small molecule, or DNA recognition has not been explored fully. As noted below, examples of fabricated glycan arrays in accordance with principles of the present invention have been made using carbohydrate-bearing brush polymers for glycan arrays. The Polymer Pen Lithography (PPL) induced or Beam Pen Lithography (BPL) induced surface initiated method could be used to site-specifically immobilize biomolecules and optimize the density of active groups. Polymer gradients with linear variation in grafting would regulate cell adhesion. If different materials can be printed in different positions, then this would provide a combinatorial method to probe both chemical structure (i.e. difference between glucose and mannose) or the superstructure (differences in chain length) simultaneously. Polymer brushes also can control friction locally or be used for self-cleaning materials. In one implementation, the methods and systems exhibit a linear growth rate.

### General Methods

Cy3-labelled ConA was purchased from Protein Mods (USA). All solvents and reagents were purchased from Aldrich or VWR and used without further purification unless otherwise noted. All solvents were dried prior to use. Solutions were prepared from nanopure water purified from Milli-Q plus system (Millipore Co.), with a resistivity over 18 MΩ cm⁻¹. Thin-layer chromatography was carried out using aluminum sheets pre-coated with silica gel 60 (EMD 40-60 mm, 230-400 mesh with 254 nm dye). All reactions were carried out under an inert atmosphere of N₂ using standard Schlenk techniques or an inert-atmosphere glovebox unless otherwise noted. Deuterated solvents were purchased from Cambridge Isotope Laboratories Inc. and used as received. NMR spectra were obtained on a Bruker ADVANCE 400 MHz spectrometer. All chemical shifts are reported in ppm units with reference to the internal solvent peaks for ¹H and ¹³C chemical shifts, and all spectral data were consistent with their reported literature values. High-resolution mass spectrometry analyses were carried out on an Agilent 6200 LC/MSD TOF system. Compounds 1 (Mehlich, J.; Ravoo, B. J. Org. Biomol. Chem. 2011, 9, 4108.), compound 2 (Mehlich, J.; Ravoo, B. J. Org. Biomol. Chem. 2011, compound 9, 4108.), compound 4 (Ruiz, J. R. J.; Osswald, G.; Petersen, M.; Fessner, W. D. J. Mol. Catal., B Enzym. 2001, 11, 189.), compound 5 (Cumpstey, I.; Butters, T. D.; Tennant-Eyles, R. J.; Fairbanks, A. J.; France, R. R.; Wormald, M. R. Carbohyd. Res. 2003, 338, 1937.) and compound 6 (Pittman, C. U.; Voges, R. L.; Jones, W. R. Macromolecules 1971, 4, 291.) were prepared according to published literature procedures. 3 α-glucomethacrylate prepared according to well known methods. The described methods may are generally applicable for for methacrylates with nearly any substituent, including any saccharide.

Compound 7 (Ferrocene alkene) was prepared as follows. HATU (184 mg, 0.48 mmol), 3-butenamine hydrochloride (52 mg, 0.48 mmol), and DIPEA (338 5 L,1.93 mmol) were added to the stirring solution of ferrocene carboxylic acid (111 mg, 0.48 mmol) in 50 mL dry DMF and was subsequently stirred for 16 h under N₂. EtOAc (50 mL) was added to the solution and the reaction mixture was washed with 50 mL saturated solution of NH₄Cl, NaHCO₃, and NaCl. Column chromatography (SiO₂, 1:1::EtOAc:Hexane) and evaporation of the solvent afforded compound 7 as yellow solid (80 mg, 57%). ¹H NMR (400 MHz, CDCl₃): δ 5.77 (m, 2H), 5.08 (m, 2H), 4.55 (t, J=1.8 Hz, 2H), 4.24 (t, J=1.9 Hz, 2H), 4.10 (s, 5H), 3.40 (q, 2H, J=9.3 Hz), 2.27 (q, J=8 Hz, 2H) ppm. ¹³C NMR (400 MHz, CDCl₃): δ 170.11, 135.63, 117.28, 70.30, 69.73, 68.02, 38.32, 34.13 ppm. HRMS m/z calcd for C₁₅H₁₈FeNO (MH⁺): 284.0738. Found: 284.0742.

### Monolayer Preparation

Microscope glass slides were purchased from VWR. 11-Amino-1-undecanethiol was purchased from Dojindo Molecular Technologies, Inc. N-Succinimidyl-S-acetylthiopropionate (SATP) was purchased from Thermo Scientific, Inc. All other chemicals and materials were purchased from Aldrich, and all chemicals were used as received. Metals were evaporated using Bal-Tec MED 020 Coating System.

To prepare Thiol-terminated Au surfaces, glass slides were coated with thermally evaporated 5 nm Cr and 100 nm Au after sonication in EtOH for 20 min. The resulting Au slides were immersed in 1.0 mM 11-mercapto-undecanamine hydrochloride (MUAM) for 24 h. The resulting amine surface was covered with 2.0 mM SATP in 10% DMF and 90% 0.1 M TEA buffer solution (pH 7.0). Then the surface was immersed in a solution of 0.5 M hydroxylamine, 0.05 M DTT, 0.05 M phosphate buffer, and 0.025 M EDTA at pH 7.5 for 20 min.

To prepare Thiol-terminated glass surfaces, glass slides were cleaned by sonication in 1M HNO₃, H₂O and EtOH for 15 min each. After drying under a N₂ stream, the glass slides were incubated in a 20 mL vial containing 0.8 mL (3-Mercaptopropyl)trimethoxysilane (3-MPTS) in 18 mL toluene at 37° C. with gentle agitation for compound 4 h. Then the glass samples were removed and rinsed thoroughly with toluene, EtOH/toluene (1:1), and absolute EtOH. Finally the samples were cured in oven at 105° C. for 18 h and were stored in MeOH at compound 4° C. until used.

To prepare alkene-terminated glass surfaces, glass slides were cleaned by sonication in pentane, Me₂CO, and H₂O for 15 min each and subsequently immersed in a piranha solution (3:1 H₂SO₄: 30% H₂O₂ (aq)) for 30 min. After washing thoroughly with H₂O and dried with an N₂ stream, the surfaces were incubated in a stirred toluene solution containing 0.10% 10-undecenyl trichlorosilane for 2 h. Finally the alkene-terminated glass surfaces were washed with EtOH and H₂O.

### Polymer Pen Lithography

To prepare the pen arrays for inking, they were exposed to O₂ plasma (Harrick PDC-001, 30 s, medium power) to render the surfaces of the pen-arrays hydrophilic. Then 4 drops of the ink solution, comprised of the thiol, acrylate, methacrylate, or alkene ink (0.80 mg), PEG (2000 g mol⁻¹, 5 mg mL⁻¹) and DMPA (0.30 mg, 1.17 mmol) in 1 mL 80:20 THF:H₂O that was sonicated to ensure solution homogeneity, were spin coated (2000 rpm, 2 min) onto the PPL pen array. A Park XE-150 scanning probe microscope equipped with a PPL head (Park Systems Corp.), XEP custom lithography software, and an environmental chamber capable of controlling humidity were used for PPL writing at a humidity of 78%-82% at room temperature. The tip array was leveled by optical methods or force methods with respect to the substrate surface using an xy tilting stage. Then the tip arrays were printed onto the thiol- or alkene-terminated glass surface into dot patterns with dwell-times varying from 50-100 000 ms at 80-85% humidity. The surface was placed under the UV light (3 mW/cm²) for 3 h and then washed thoroughly with 50 ml EtOH and H₂O.

### Fluorescence Microscopy

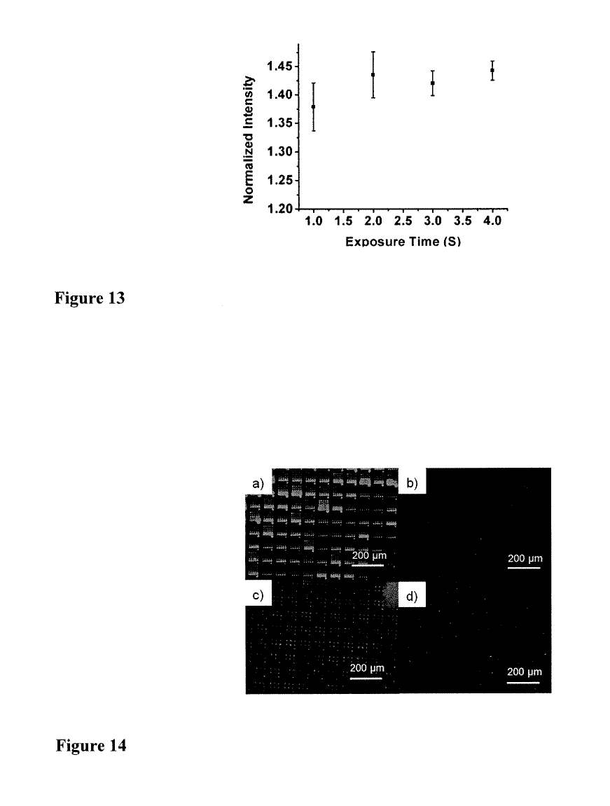

Fluorescence intensity profiles of the array were obtained from a Nikon Eclipse Ti fluorescence microscope (λₑₓ=532-587 nm, λ_{obs}=608-683 nm), and extracted by NIS-elements software (Nikon Instruments, Inc.). Exposure times ranged from 1-4 s, depending on the brightness of the arrays. To compare data taken with different exposure times, the normalized fluorescence was obtained by dividing the maximum fluorescence by the background fluorescence. It is possible that the ratio of signal-to-background can vary with exposure time, so to test the effect of exposure time on the normalized fluorescence of features on the microarray, fluorescence images of a surface patterned with compound 4 and bound to Cy3-modified ConA were taken at different exposure time. It was found the normalized fluorescence in this time range is independent of the exposure time (FIG. 13).

### Control Experiments

Rhodamine-methacrylate (1) Printing on Bare Glass Surfaces and on Thiol-terminated Glass Surfaces without UV Exposure. In the first control experiment, ink mixture containing compound 1 (0.80 mg, 1.2 mmol), PEG (2000 g mol⁻¹, 5 mg mL⁻¹) and DMPA (0.30 mg, 1.17 mmol) was deposited onto a bare glass surface by PPL, and ink deposition was confirmed by fluorescence microscopy (FIG. 14a). Following UV exposure, no fluorescent pattern was observed (FIG. 14b). Alternatively, compound 1 was patterned onto the alkene terminated glass surface, and deposition was confirmed by fluorescence microscopy (FIG. 14c), and then washed with 50 mL EtOH and H₂O without exposure to UV light. Some dim patterns could still be seen on the alkene terminated glass surface even without UV light (FIG. 14d).

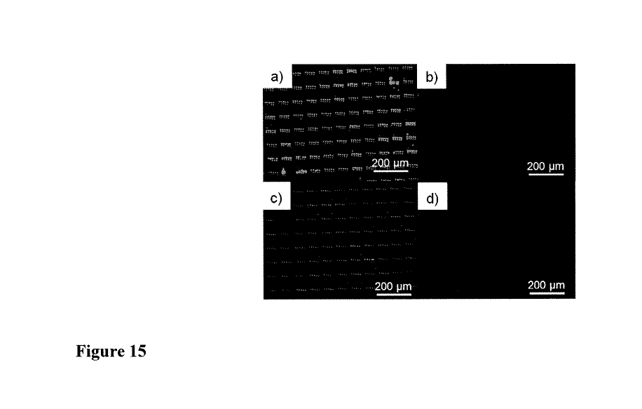

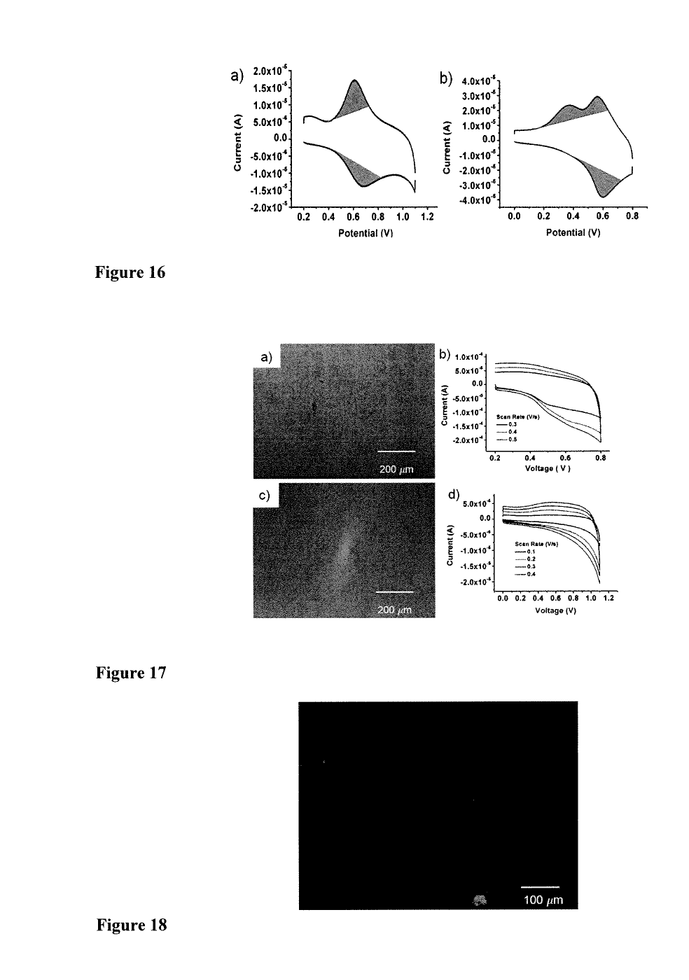

Rhodamine-thiol (2) Printing on Bare Glass Surfaces and on Alkene-terminated Glass Surfaces without UV Exposure. In the first control experiment, ink mixture containing compound 2 (0.80 mg, 1.2 mmol), PEG (2000 g mol⁻¹, 5 mg mL⁻¹) and DMPA (0.30 mg, 1.17 mmol) was deposited onto a bare glass surface by PPL, and ink deposition was confirmed by fluorescence microscopy (FIG. 15a). Following UV exposure, no fluorescent pattern was observed (FIG. 15b). Alternatively, compound 2 was patterned onto the alkene terminated glass surface, and deposition was confirmed by fluorescence microscopy (FIG. 15c), and then washed with 50 mL EtOH and H₂O without exposure to UV light. Some dim patterns could still be seen on the alkene terminated glass surface even without UV light (FIG. 15d).

### Atomic Force Microscopy of α-Glucomethacrylate 5

Compound 5 was patterned onto the thiol-terminated glass surface by PPL, as described above, with a dwell time of 3 s, irradiated with UV light for 3 h, and washed with H₂O and EtOH to remove any unreacted ink. AFM characterization of the height profile of the features on the surface patterned with compound 5 after washing was performed on a Park XE-150 Scanning probe microscope (Park Systems Corp.) using Nanosensors™ PPP NCHR probes under non-contact mode. The feature heights of the patterns produced by PPL were measured using the AFM topographic height profile.

### Cyclic Voltammetry

A custom built bored Teflon cone (7 mm inner diameter) was pressed against the gold surface. 0.1M HClO₄ (aq) electrolyte solution was added to the bore. A Pt counter electrode and a glass frit-isolated Ag/AgCl reference electrode were used in the measurement of the study. An electrochemical workstation (CH Instruments, Inc., CHI 440) was used to control the potential and convert the cell current to a potential signal. A Tektronix TDS 520 digital oscilloscope recorded the current response signal from the potentiostat while a Wavetek 395 function generator generated potential program signal. All measurements were conducted at room temperature.

Calculation of Fc Cover Density. The cover density of ferrocene (fc), Γ_{fc}, was calculated using Eq.

Γ_{fc}=Q_{fc}/neA (Eq. 1)

where Q_{fc}, the total charge passed in the redox reaction, was calculated by dividing the integral of the redox peak after linear base line subtraction (FIG. 17) by the corresponding scan rate. The Q_{fc} for the PPL deposited compound 6 and compound 7 was (6.48±0.09)×10⁻⁶ and (1.79±0.08)×10⁻⁶ C respectively. A, the surface area of the working electrode, was calculated by the total area covered by compound 6 and compound 7. For the PPL deposited compound 7, A=0.103 cm². So Γ_{fc} for the PPL deposited compound 7 was calculated as (7.22±0.12)×10¹⁴ cm⁻². For the PPL deposited 6, A=0.0437 cm². So Γ_{fc} for the PPL deposited compound 6 was calculated as (7.31±0.25)×10¹⁵ cm⁻².

Cyclic Voltammetry Control Experiments. In the first control experiment, ink mixture containing compound 6 (0.80 mg, 1.2 mmol), PEG (2000 g mol⁻¹, 5 mg mL⁻¹) and DMPA (0.30 mg, 1.17 mmol) was deposited onto thiol-terminated glass surface by PPL without UV exposure and following identical procedure described above. Prior to washing, ink transfer was confirmed by optical microscopy (FIG. 18a). Following washing, no ferrocene signals were observed on the bare glass surface (FIG. 18b). In the second control experiment, ink mixture containing compound 7 (0.80 mg, 1.2 mmol), PEG (2000 g mol⁻¹, 5 mg mL⁻¹) and DMPA (0.30 mg, 1.17 mmol) was deposited onto pure Au surface by PPL with UV exposure and following identical procedure described above. Prior to washing, ink transfer was confirmed by optical microscopy (FIG. 18c). Following washing, no ferrocene signal was observed on the bare glass surface (FIG. 18d).

### Beam Pen Lithography

Polymer Brushes of Rhodamine-methacrylate (1) on Thiol-terminated Glass Surfaces were prepared. To prepare the pen arrays for inking, they were exposed to O₂ plasma (Harrick PDC-001, 30 s, medium power) to render the surfaces of the BPL pen-arrays hydrophilic. Then 4 drops of the ink solution, comprised of compound 1 (0.80 mg, 1.2 mmol), PEG (2000 g mol⁻¹, 5 mg mL⁻¹) and DMPA (0.30 mg, 1.17 mmol) in 1 mL 80:20 THF:H₂O that was sonicated to ensure solution homogeneity, were spin coated (2000 rpm, 2 min) onto the pen BPL array. A Park XE-150 Scanning probe microscope equipped with a PPL head (Park Systems Corp.), XEP lithography software, and an environmental chamber capable of controlling humidity were used for BPL writing at a humidity of 66%-72% at room temperature. The tip array was leveled by optical methods with respect to the substrate surface using an xy tilting stage. The ink mixture was patterned into dot arrays with 1 s dwell time. After printing, AFM-controlled BPL tip array was brought back to each spots and exposed for different times (2 min, 5 min, 10 min, 20 min) under the 365-nm UV lamp (46.7 mW), and the substrate was subsequently washed and sonicated with 50 ml EtOH and H₂O.

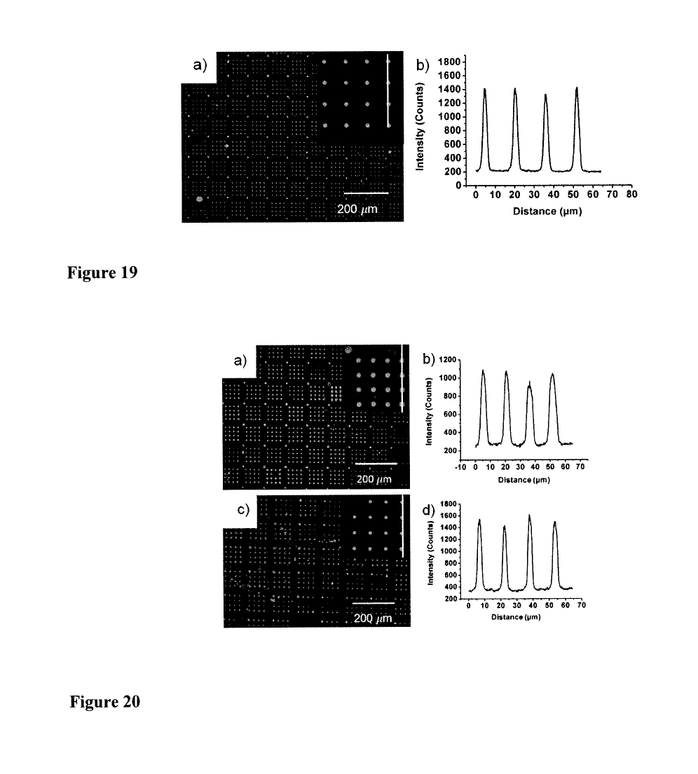

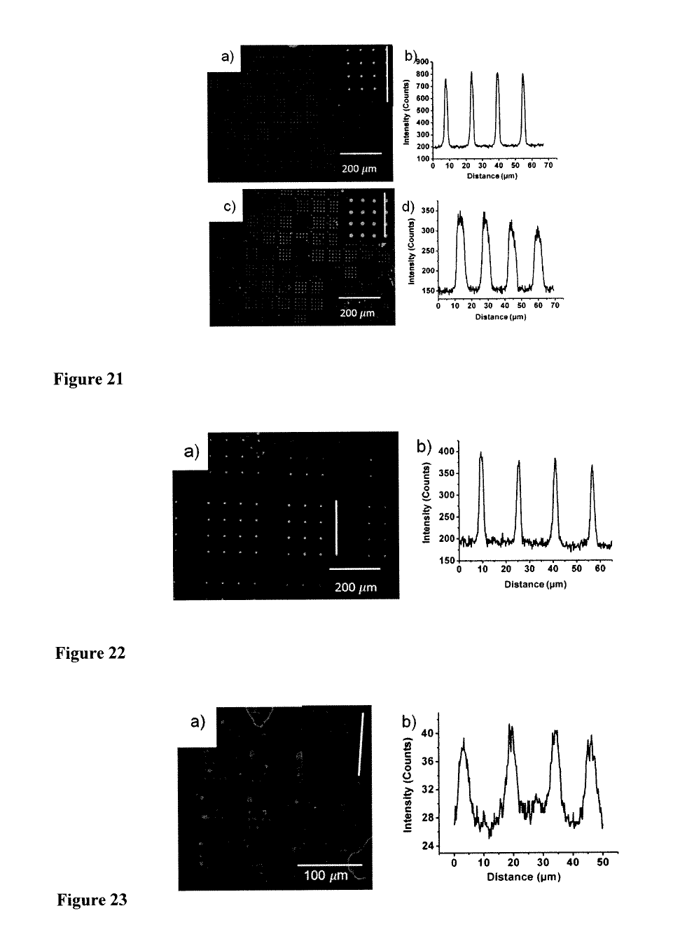

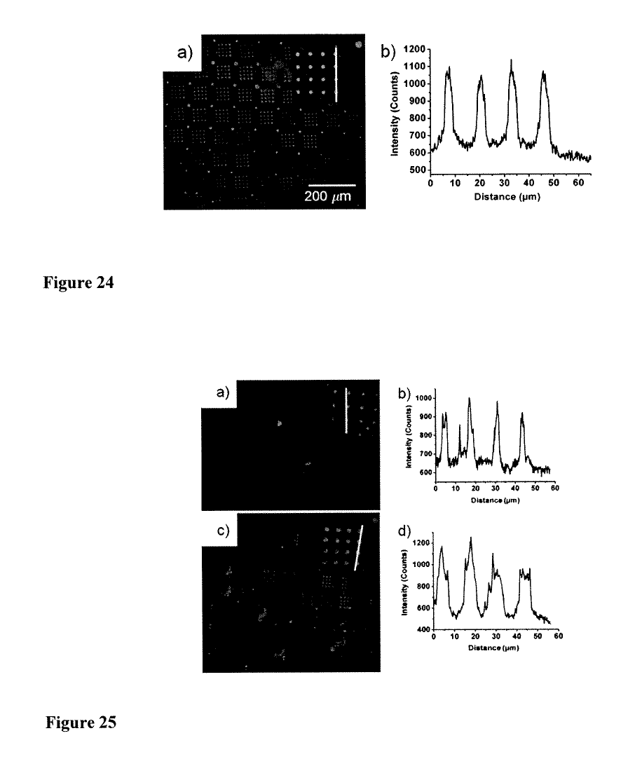

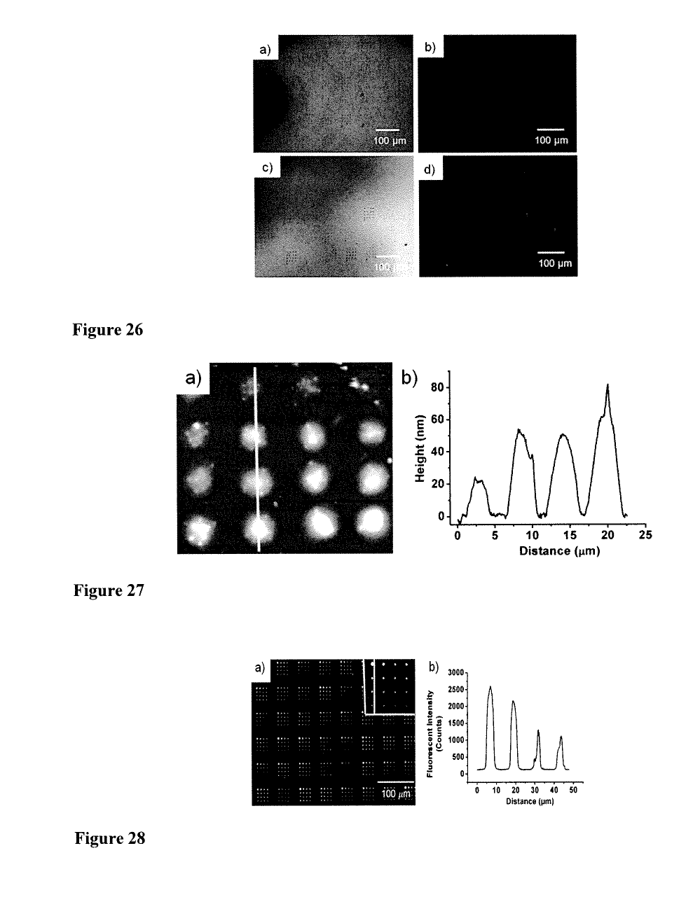

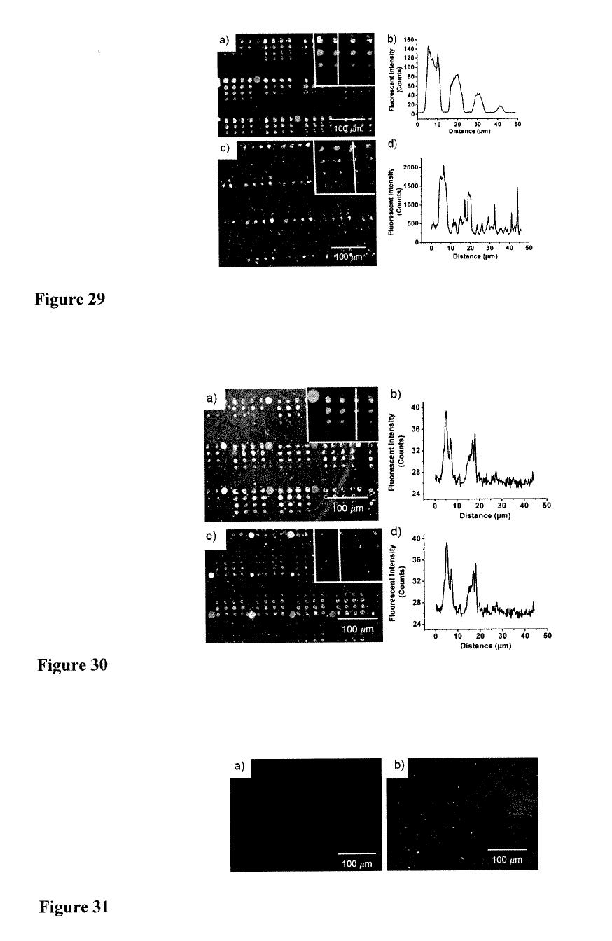

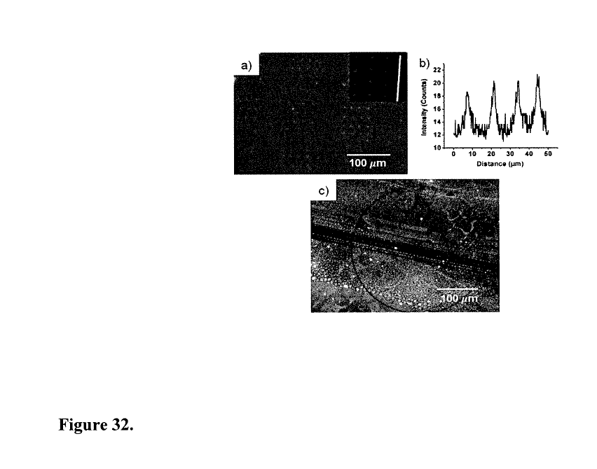

Control Experiments. In the control experiment, ink mixture containing compound 1 (0.80 mg, 1.2 mmol), PEG (2000 g mol⁻¹, 5 mg mL⁻¹) and DMPA (0.30 mg, 1.17 mmol) was deposited onto a thiol-terminated glass surface by BPL without UV exposure and following identical procedure described above. No fluorescent patterns were observed on the bare glass surface (FIG. 19).

### Lectin Binding

Preparation of Carbohydrate arrays by PPL-induced Thiol-ene and Thiol-acrylate reaction. To prepare the pen arrays for inking, they were exposed to O₂ plasma (Harrick PDC-001, 30 s, medium power) to render the surfaces of the pen-arrays hydrophilic. Subsequently 4 drops of the ink solution, comprised of 5 (2.18 mg, 10 mM), PEG (2000 g mol⁻¹, 5 mg mL⁻¹) and DMPA in 1 mL 80:20 THF:H₂O that was sonicated to ensure solution homogeneity, were spin coated (2000 rpm, 2 min) onto the pen PPL array. A Park XE-150 Scanning probe microscope equipped with a PPL head (Park Systems Corp.), XEP lithography software, and an environmental chamber capable of controlling humidity were used for PPL writing at a humidity of 78%-83% at room temperature. The tip array was leveled by optical methods with respect to the substrate surface using an xy tilting stage. After placed under the UV light (3 mW/cm²) for 6 hours, the slide was washed with 30 ml THF and 30 ml H₂O and dried with N₂. Then the slide was immersed in bovine serum albumin (1%) solution for 2 hours and washed 3 times with aqueous phosphate buffer (10 mM, pH 7.4, 0.005% Tween 20). After drying with N₂, the slide was immersed in Cy3-ConA solution of varying concentrations (21.7×10⁻⁶, 10.8×10⁻⁶, 5.4×10⁻⁶, 3.0×10⁻⁶, 1.7×10⁻⁶, 4.3×10⁻⁷, 2.1×10⁻⁷ M, Figures S19-S25) for 5 h at 4° C., washed 3 times with aqueous phosphate buffer (10 mM, pH 7.4, 0.005% Tween 20), and dried with N₂. The carbohydrate arrays of 6 and 7 were prepared following the same procedure.

Control Experiments. Compound 3 and compound 4 were printed following the aforementioned procedure and immersed in a solution of Rhodamine-labeled PNA (1.5×10⁻⁵ M), which is a galactose-specific lectin that does not bind mannose or glucose, for 5 h. Following washing with aqueous phosphate buffer (10 mM, pH 7.4, 0.005% Tween 20), three times, no visible fluorescence was observed. This experiment supports the conclusion that fluorescence in the Cy3-labelled ConA exposed arrays of compound 3 and compound 4 arise from ConA-glucose binding (FIG. 27).

### Example Implementations

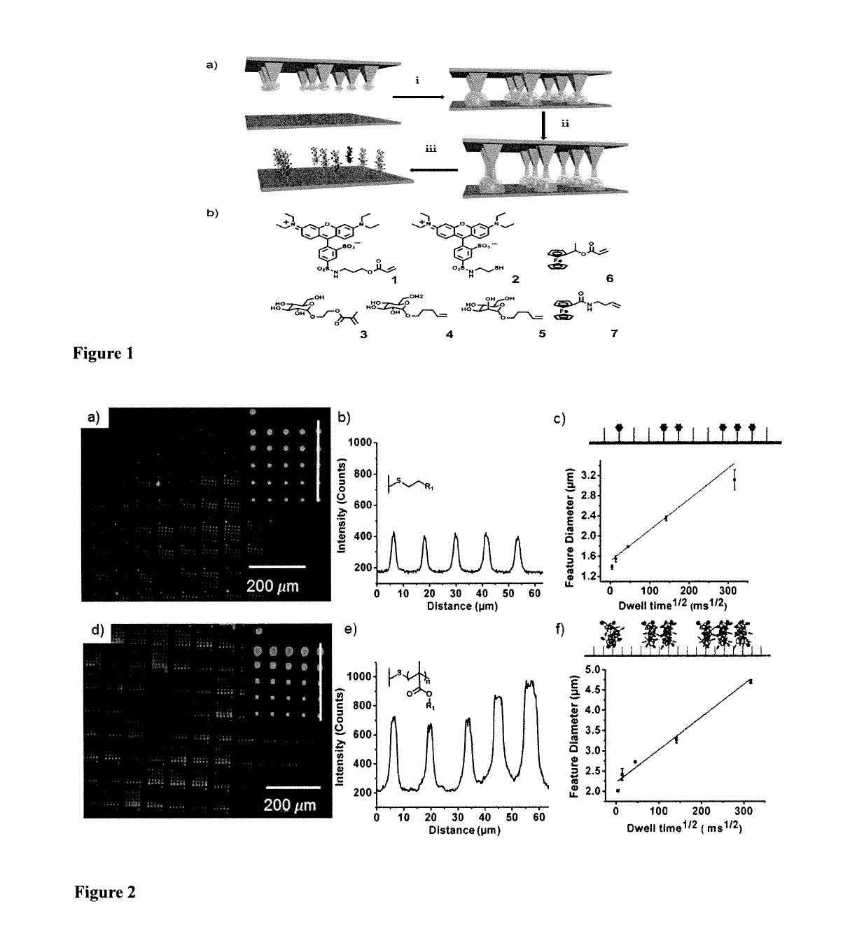

Patterns were created by either depositing an ink mixture containing the methacrylate monomers and 2,2-dimethoxy-2-phenylacetophenone (DMPA) photoinitiator by polymer pen lithography (PPL) and subsequently irradiating the surface or using beam pen lithography (BPL) (FIG. 1a) to deposit the ink and control precisely the irradiation time of each feature in the array. To confirm this covalent reaction occurred on surfaces and quantify ligand density, fluorescent compound 1 and redox-active compound 6 inks were patterned on glass and Au surfaces, respectively, and compared to results obtained with fluorescent compound 2 and redox-active compound 7 alkenes, which instead form monolayers by the thiol-ene reaction. We found that the average oligomer length of the polymer brushes can be controlled by varying the irradiation time. Finally, binding studies between arrays of α-glucoside compound 3 or α-mannoside compound 5 monolayers with GBPs confirmed that a significant increase in detection sensitivity occurs as a result of the multivalency of the brush polymers prepared by the thiol-acrylate polymerization compared to monolayers prepared by the thiol-ene reaction.

Molecular probes compounds 1-7 (FIG. 1b) were purchased from commercial sources or synthesized and characterized by ¹H NMR, ¹³C NMR, and high resolution mass spectrometry. PPL is a molecular printing method that uses elastomeric arrays with as many as 10⁷ nanoscopic tips mounted onto the piezoelectric actuators of an atomic force microscope (AFM) to deposit inks in arbitrary patterns on a surface. Because patterns are formed by transfer of ink through an aqueous meniscus, precise feature size control from 80 nm to 100 μm is achieved by varying the dwell time or force between the tip arrays and the surface, and PPL has been used successfully to create functional arrays of biological probes. To create fluorescent patterns by PPL, 8,500 tip PDMS arrays with a tip-to-tip spacing of 80 or 160 μm were prepared following previously published literature protocols. The photoreaction between methacrylate or alkene inks and the thiol- or alkene-terminated surface was induced by spin coating (2000 rpm, 2 min) the dye (0.8 mg), DMPA (0.33 mg) and poly(ethylene glycol) (PEG) (2000 g mol⁻¹, 5 mg ml⁻¹) in 0.8 mL 60:20 THF:H₂O, which was sonicated to ensure solution homogeneity, onto the tip array. The PEG matrix that encapsulates the inks ensures even ink distribution across the tip array and reproducible ink transport from the tips to the surface. The tips were mounted onto the z-piezo of an AFM that was specially equipped to hold the tip arrays, an environmental chamber to regulate the humidity, and customized lithography software to control the position and dwell-time of the tips. Patterns were created by repeatedly bringing the tip arrays into contact with the surface with dwell times ranging from 50 to 100,000 ms and subsequently irradiating the surface with UV light (λ=365 nm, intensity=3 mW/cm²).

Fluorescence microscopy images confirmed the successful immobilization of inks compound 1 (Rhodamine-methacrylate) and compound 2 (Rhodamine-thiol) by the PPL-induced thiol-acrylate and thiol-ene reactions, respectively with feature diameter control. Inks compound 1 and compound 2 were patterned onto thiol-terminated and alkene-terminated glass slides, respectively, by PPL, exposed to 365 nm light for 3 h, and the surfaces were subsequently washed with EtOH and H₂O. While both inks produced fluorescent patterns, the differences in normalized fluorescence intensities indicated the formation of polymers when methacrylate, rather than thiol, inks are used. The normalized fluorescence of patterns produced with compound 2 (FIGS. 2a and 2b) was (1.9±0.1), which lies within the range of 1.4-1.9 that is consistent with previous observations for monolayer coverage of fluorophores irrespective of the microscope. However, patterns prepared under identical conditions with compound 1 that is capable of polymerizing had a normalized fluorescence of (3.6±0.2) (FIGS. 2c and 2d). We attribute this increase in fluorescence signal to the formation of brush polymers protruding from the surface that are side chain functionalized with the Rhodamine dye, which increases fluorescence intensity above the value than could be obtained by monolayer coverage alone. In both cases, feature diameter increases linearly with dwell time.

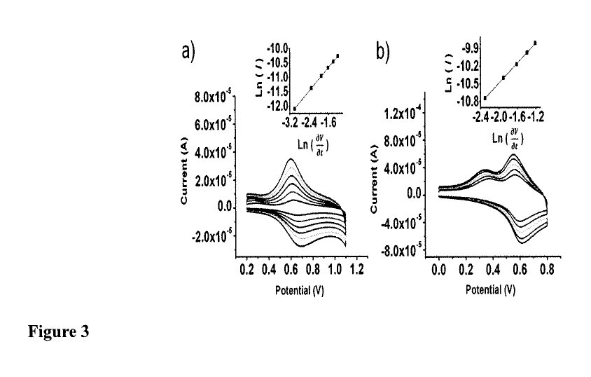

Electrochemically active acrylate compound 6 and alkene compound 7 were patterned onto thiol-terminated Au surfaces by the protocol described above, and deposition of the inks was confirmed by optical microscopy. Following washing of the surface with EtOH and H₂O and sonicating in THF for 3 min to remove the PEG and unreacted inks, cyclic voltammetry (CV) was carried using a Ag/AgCl reference electrode, a Pt counter electrode, and the patterned Au surfaces as the working electrodes. Strong redox peaks at E^{o}=556 mV and 611 mV (vs. Ag/AgCl) for compound 6 (FIG. 3b) and compound 7 (FIG. 3a), respectively, confirmed the presence of the ferrocene (fc)/ferrocenium (fc⁺) reversible redox couple. Control experiments, where inks were deposited but not treated with light or deposited onto surfaces that were not functionalized with thiols, did not result in any observable current corresponding to the fc/fc⁺ redox couple after washing, confirming that light is necessary to immobilize the inks. The linear relationship between peak current and scan rate

$$\frac{\partial V}{( \partial t )}$$

confirmed that compound 7 is immobilized on the Au surface in monolayer coverage, and that the measured charge density (Γ_{fc}) of (7.22±0.12)×10¹⁴ cm⁻² is similar to the value expected for a densely packed fc monolayer. The CV of surfaces patterned with compound 6, differ significantly in ways strongly indicative of the formation of polymers: the presence of broadened oxidation peaks which is indicative of heterogeneity within polymeric features, the increase in ΔE with

$$\frac{\partial V}{\partial t} ,$$

and the slope of 0.7 in the ln

$$\frac{\partial V}{( \partial t )}$$

vs. ln(l) which indicates complexity in the charge transfer from the fc to the Au consistent similar to hopping in conductive polymers. From integration of the CV, a Γ_{fc} of (6.25±0.06)×10¹⁵ cm⁻² was obtained, which is an order of magnitude higher than was obtained from compound 7, suggesting a degree of polymerization of ˜10. Thus both CV and fluorescence data are consistent with a surface oligomerization of methacrylate monomers from a thiol-terminated surface.

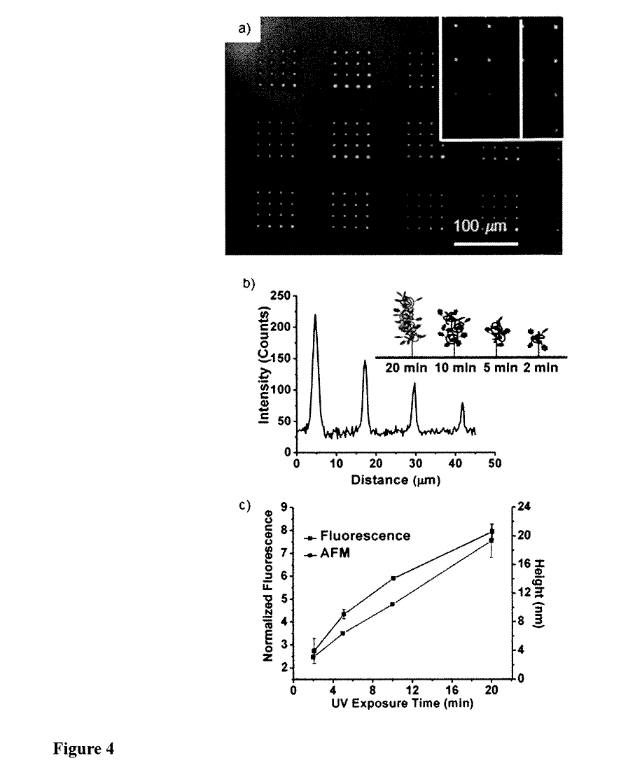

To determine whether chain length could be controlled in situ, compound 1 was also patterned onto thiol-terminated glass surfaces by BPL tip arrays following the conditions described above. Two important capabilities of BPL are that the tips can return to the position where ink was deposited, and the irradiation time at each position in an array can be controlled precisely. To test how irradiation time affected chain lengths, an ink mixture composed of compound 1, PEG, and DMPA was spin coated onto a BPL array, and a 4×4 dot array was printed with dwell-times of 1 s at each spot. Using the nanoscale precision of the piezoactuators that control the movements of the BPL tip arrays, the tips were held 5 μm above the dots patterned onto the surface, and different spots in each array were irradiated for times of 2, 5, 10 and 20 min (λ=365 nm, intensity=46.7 mW). The surfaces were then washed with EtOH and H₂O, sonicated in THF for 5 min, and the resulting arrays were imaged by fluorescence microscopy (FIG. 4a). Fluorescence intensity increased linearly with irradiation time (FIG. 4b), confirming that chain-length can be controlled in situ by a chain-growth polymerization mechanism, and because this is not a living polymerization, constant irradiation is necessary for polymer growth, thereby enabling precise control over feature size. It should be noted that while-thiols are well known chain-transfer agents and is likely occurring in these films, chain transfer should not affect the polymer growth rate.

An advantage of the thiol-acrylate surface photopolymerization is that multivalent glycan-bearing brush polymers can be prepared in a single process. Arrays of compound 3 and compound 4 were prepared by the thiol-methacrylate and thiol-ene reactions, respectively, as well as α-mannosides immobilized by the thiol-ene and Cuaac reaction. Binding of Concanavalin A (ConA) to these arrays was measured by fluorescence microscopy to determine how the mutivalency of the resulting glycans affects binding. ConA is a mannose-specific GBP (Kₐ=10³-10⁴ M⁻¹) that also binds glucosides, albeit weakly (Kₐ˜10² M⁻¹). However, in solution and on surfaces the binding of ConA with multivalent glycan scaffolds increases significantly because of the four identical carbohydrate recognition domains on the protein compound 3 was patterned onto the surface by PPL, irradiated for 3 h, and washed with H₂O and EtOH to remove any unreacted ink, resulting in glycan features with average heights of 16±3 nm. The surfaces were subsequently immersed in a solution of 1% BSA to passivate any unreacted thiols on the surface and subsequently washed with PBS-Tween 20 (0.01 M PBS, 0.005% Tween 20, pH 7.4) and PBS (0.01 M PBS, pH 7.4) solutions.

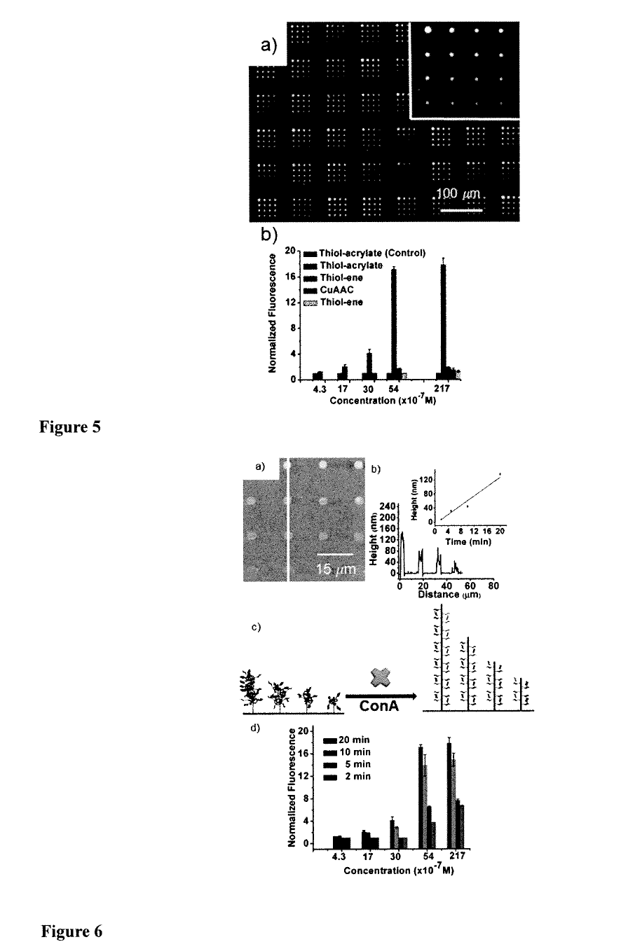

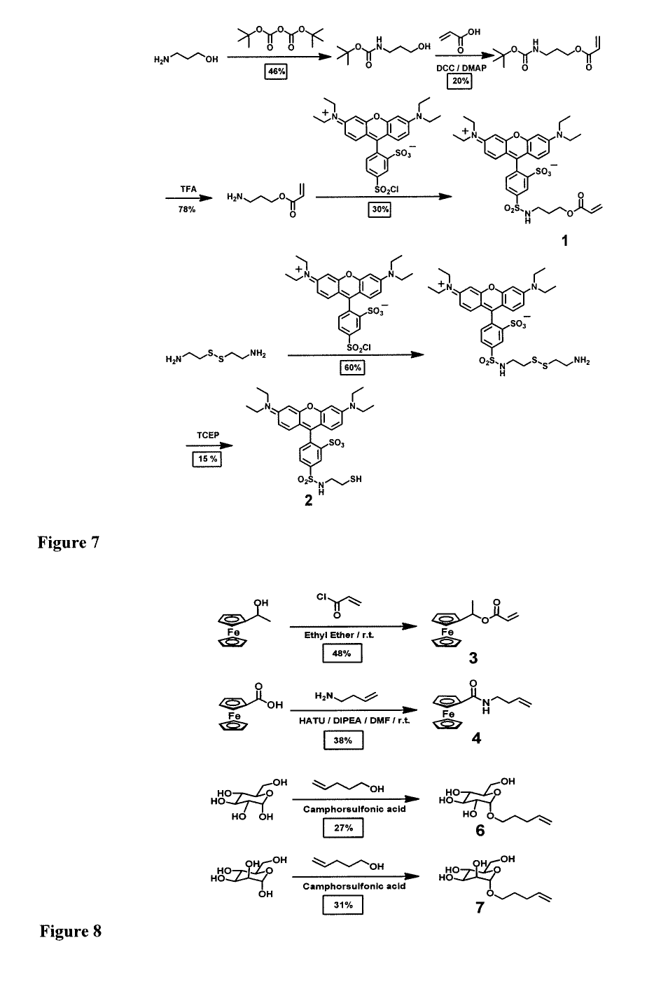

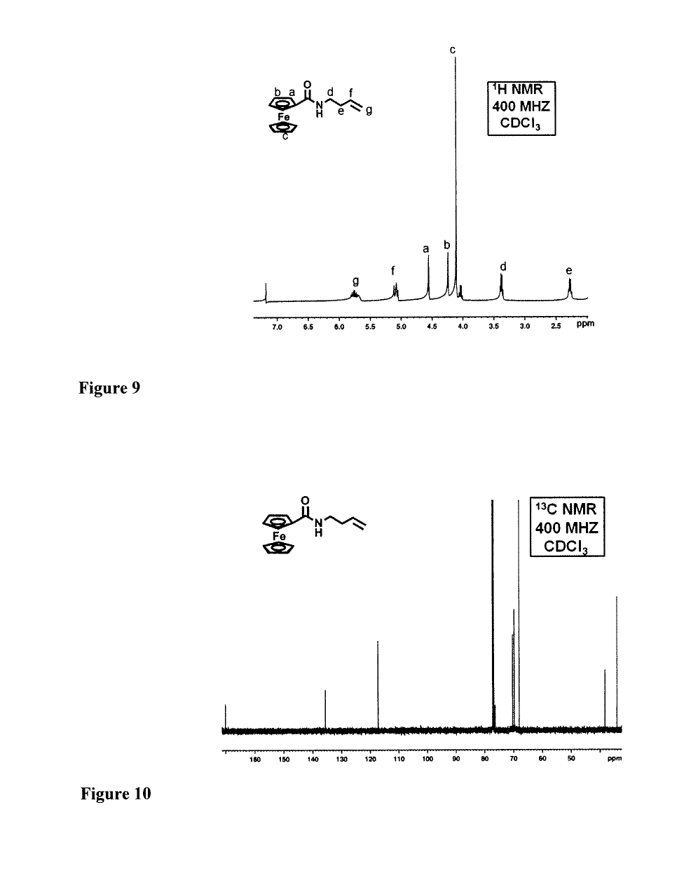

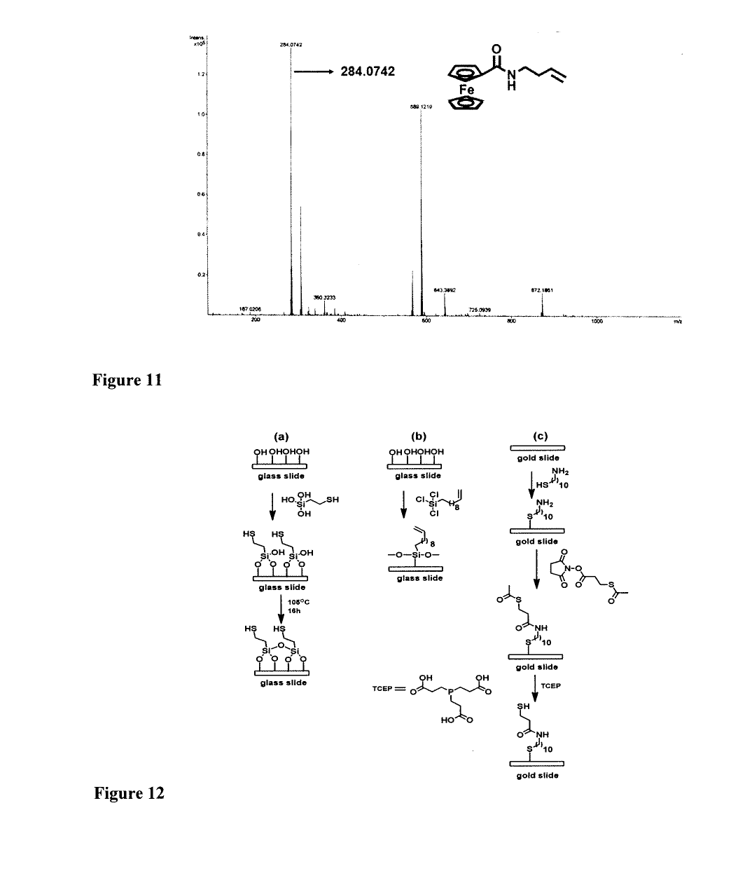

To assay binding against ConA the substrates were immersed in a buffered solution of Cy3-labelled ConA (0.5 mg/mL) for 4 h at 4° C. The surfaces were then washed with PBS-Tween 20 and PBS solutions to remove unbound protein, and the binding of the fluorescently labeled ConA to the glycans in the arrays were determined by fluorescence microscopy (FIG. 5). While the signal for arrays prepared with compound 4 was only slightly above the noise level (normalized fluorescence=1.20±0.12), which is consistent with the expected low binding between ConA and glucosides, arrays of oligomers of compound 3 had a normalized fluorescence of (7.1±0.2). By contrast monolayers of mannosides prepared by the thiol-ene reaction or the Cu^{I}-catalyzed azide alkyne click reaction only provided normalized fluorescence of 1.4 and 1.6, respectively, under identical conditions. The concentration of ConA was varied systematically (FIG. 5), and ConA could be detected by arrays of compound 3 at concentrations as low as 0.2 μM, while monolayers of compound 4 did not display any signal below 0.5 mg/mL. As a control, the glycan arrays of compound 3 and compound 4 were exposed to Rhodamine-modified PNA, which is a GBP that does not bind glucosides, and no fluorescence was observed, confirming that the observed fluorescent patterns were the result of specific glucose-ConA recognition. We attribute this incredible sensitivity of arrays of these 3D brush polymers to the cluster-glycoside effect, which is an enhancement of affinity of multimeric carbohydrate scaffolds compared to their monovalent counterparts.

Site-specific thiol-acrylate photopolymerization can create microarrays composed of multivalent brush polymers with well-defined 3D structures. Fluorescence and electrochemical studies confirmed the oligomeric nature of the immobilized molecules and that chain length can be controlled by varying the illumination time. The utility of this chemistry was demonstrated by creating glycan-bearing brush polymer arrays, whose high sensitivity towards GBPs arises from the cluster-glycoside effect. This new method of creating biological microarrays uses easy-to-prepare acrylate and methacrylate monomers to form complex 3D nanostructures, and we anticipate that this functional-group tolerant chemistry could become a popular method for preparing glycan microarrays. Future work will focus on elucidating the structure of the polymers, studying reaction kinetics, and on creating multicomponent glycan structures.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A microarray comprising:
a thiol-terminated substrate;
a plurality of polymer brushes bound via thiol-(meth)acrylate polymerization to the thiol-terminated substrate.

2. The microarray of claim 1, wherein the polymer brushes exhibit a linear length growth rate from the substrate.

3. The microarray of claim 1, wherein the polymer brushes comprise a polymer grown by free radical polymerization.

4. The microarray of claim 1, wherein the polymer brushes comprise a materials selected from n (meth)acrylate oligomer and poly((meth)acrylate polymer.

5. The microarray of claim 1, wherein each of the polymer brushes comprise a plurality of binding sites for molecules selected from the group consisting of glycans, glycan binding proteins, antibodies, peptides, small molecules, and DNA.

6. A method of making a microarray comprising:
providing a substrate having thiol associated therewith;
depositing (meth)acrylate-containing monomers on the substrate;
depositing an initiator on the substrate;
irradiating the substrate with the deposited (meth)acrylate-containing monomers and photoinitiator; and
inducing thiol-(meth)acrylate polymerization;
wherein an oligomer comprising the (meth)acrylate-containing monomers and a thiol-acrylate is bound to the substrate.

7. The method of claim 6, wherein the initiator is selected from the group consisting of a photoinitiator or a radical initiator.

8. The method of claim 7, wherein the initiator is a photoinitiator.

9. The method of claim 8, wherein the deposition of the (meth)acrylate-containing monomers and deposition of the photoinitiator is done simultaneously.

10. The method of claim 8, wherein the deposition of the (meth)acrylate-containing monomers and deposition of the photoinitiator is by polymer pen lithography and further wherein the acrylate-containing monomers and the photoinitiator are constituents of an ink for polymer pen lithography.

11. The method of claim 8, wherein the irradiation is by beam pen lithography.

12. The method of claim 8 wherein the photoinitiator is selected from the group consisting of 2,2-dimethoxy-2-phenylacetophenone (DMPA), benzoyl peroxide (BPO), and AIBN (azobisisobutyronitrile).

13. The method of claim 8, further comprising selectively controlling irradiation time wherein average oligomer length is controllable.

14. The method of claim 6, wherein the deposition is by a method selected from microcontact printing and dip-pen nanolithography.

15. A method of investigating molecules comprising:
providing a substrate having thiol associated therewith;
depositing a molecule having a (meth)acrylate functional group on the substrate;
inducing thiol-acrylate polymerization; and
forming a plurality of polymer brushes bound to the substrate;
wherein the plurality of polymer brushes provide sufficient glycan density to access multivalent glycan binding protein modes.

16. The method of claim 15, further comprising depositing a photoinitiator on the substrate.

17. The method of claim 16, further comprising irradiating the substrate and deposited (meth)acrylate-containing monomers and photoinitiator;

18. The method of claim 15 wherein the molecule is selected from the group consisting of glycans, glycan binding proteins, antibodies, peptides, small molecules, and DNA.
